(19) United States
(12) Patent Application Publication
(10) Pub. No.: US 2021/0052438 A1
(43) Pub. Date: Feb. 25, 2021

(54) ELASTIC LAMINATE WITH MULTIPLE STRETCH ZONES AND METHOD FOR MAKING SAME

(71) Applicant: TREDEGAR FILM PRODUCTS LLC, N. Chesterfield, VA (US)

(72) Inventors: Amir MOSHE, N. Chesterfield, VA (US); Stephen D. BRUCE, Montpelier, VA (US); Todd R. SKOCHDOPOLE, Moseley, VA (US); Meredith W. ALLIN, N. Chesterfield, VA (US); Tiffany Cassidy HULTQUIST, N. Chesterfield, VA (US)

(21) Appl. No.: 16/999,177

(22) Filed: Aug. 21, 2020

Related U.S. Application Data
(60) Provisional application No. 62/890,470, filed on Aug. 22, 2019.

(51) Int. Cl. A61F 13/49

(52) U.S. Cl. CPC A61F 13/4902; B32B 5/022; A61F 13/49011

(57) ABSTRACT

A method for manufacturing an elastic laminate includes conveying an elastic laminate precursor material comprising an elastic film layer and a nonwoven layer in a machine direction to an activation station, activating, at the activation station, a first zone of the elastic laminate precursor material to create a first stretch zone of the elastic laminate, and activating a second zone of the elastic laminate precursor material to create a second stretch zone of the elastic laminate having at least one stretch property different from the first stretch zone of the elastic laminate.

### CROSS-REFERENCE TO RELATED APPLICATIONS

This application claims the right of priority to U.S. Provisional Patent Application Ser. No. 62/890,470, filed Aug. 22, 2019, the entire content of which is hereby incorporated by reference.

### FIELD

The present invention relates to an elastic laminate that has multiple stretch zones and a method for making an elastic laminate with multiple stretch zones. The elastic laminate may be a component for a wearable article, such as an absorbent article.

### BACKGROUND

Elastic laminates are used in the manufacture of many goods, including wearable articles, such as garments, hats, gowns, coveralls, absorbent articles, etc., and are typically used to provide desired fit characteristics to the article. In particular, elastic laminates that are used in the manufacture of absorbent articles, such as diapers, training pants, adult incontinence articles, and similar articles help provide a close, comfortable fit about the wearer. Many conventional absorbent articles employ elastic materials in the waist section of the article in order to secure the article around a wearer. Absorbent articles may also employ various elastic configurations, such as leg cuffs, side tabs, side ears, and side panels.

Many elastic laminates known in the art include elastic strands, such as strands of LYCRA® brand elastomer, to provide elasticity to the article. In the manufacture of elastic strand laminates, the strands are placed under tension and adhesively laminated to at least one, and typically two nonwoven fibrous webs. The nonwoven webs provide a cloth like texture to the laminate. The elastic strands are then allowed to relax, causing the nonwoven to gather and pucker, resulting in a bulky appearance. In some applications, such as training pants and adult incontinence articles, the bulky appearance is objectionable. In order to make the resulting laminate smoother and less bulky, the number of elastic strands used may be increased approximately three-fold, for example. The increased number of elastic strands adds to the cost of the laminate, and also results in significantly more complicated and less robust manufacturing process. For example, the increased number of strands becomes difficult to manage and, if any of the strands break, the process may be stopped for a considerable period of time while the strand(s) are re-threaded into the machine. Moreover, laminates that include elastic strands typically provide a single, circumferentially continuous stretch zone having the same stretch properties throughout the stretch zone. Such laminates may not provide a comfortable fit for the user when the laminate is incorporated into a wearable article.

In order to provide a more comfortable fit, it is desirable to have an elastic laminate with multiple stretch zones having one or more different stretch properties.

### SUMMARY

According to an aspect of embodiments of the invention, there is provided a method for manufacturing an elastic laminate. The method includes conveying an elastic laminate precursor material comprising an elastic film layer and a nonwoven layer in a machine direction to an activation station, activating, at the activation station, a first zone of the elastic laminate precursor material to create a first stretch zone of the elastic laminate, and activating a second zone of the elastic laminate precursor material to create a second stretch zone of the elastic laminate having at least one stretch property different from the first stretch zone of the elastic laminate.

In an embodiment, the at least one stretch property is selected from the group consisting of: extensibility, modulus of elasticity, and permanent set.

In an embodiment, the second stretch zone has a level of extensibility between about 10% and about 90% of a level of extensibility of the first stretch zone. In an embodiment, the level of extensibility of the second stretch zone is between about 20% and about 80% of the level of extensibility of the first stretch zone. In an embodiment, the level of extensibility of the second stretch zone is between about 30% and about 70% of the level of extensibility of the first stretch zone.

In an embodiment, the first stretch zone and the second stretch zone extend in a direction transverse to the machine direction.

In an embodiment, the second zone of the elastic precursor material is activated at the activation station.

In an embodiment, the second stretch zone is adjacent the first stretch zone.

In an embodiment, the first stretch zone and the second stretch zone are spaced apart by a third zone in a direction transverse to the machine direction. In an embodiment, the third zone is not activated to create an inelastic zone in between the first stretch zone and the second stretch zone of the elastic laminate.

According to an aspect of the present invention, there is provided a method for manufacturing an elastic laminate. The method includes conveying an elastic laminate precursor material comprising an elastic film layer and a nonwoven layer in a machine direction to a first activation station, activating, at the first activation station, at least a portion of the elastic laminate precursor material to a first level of activation, and activating, at a second activation station downstream in the machine direction from the first activation station, at least one zone of the elastic laminate precursor material to a second level of activation greater than the first level of activation to create at least two stretch zones of the elastic laminate having at least one stretch property different from each other.

According to an aspect of the invention, there is provided an elastic laminate that includes an elastic film layer, a nonwoven layer attached to a first surface of the elastic film layer, a first stretch zone, and a second stretch zone having at least one stretch property different from the first stretch zone. In an embodiment, the at least one stretch property is selected from the group consisting of: extensibility, modulus of elasticity, and permanent set.

In an embodiment, the first stretch zone has a first level of extensibility and the second stretch zone has a second level of extensibility. In an embodiment, the second level of extensibility is between about 10% and about 90% of the first level of extensibility. In an embodiment, the second level of extensibility is between about 20% and about 80% of the first level of extensibility. In an embodiment, the second level of extensibility is between about 30% and about 70% of the first level of extensibility.

In an embodiment, the elastic laminate includes a second nonwoven layer attached to a second surface of the elastic film layer, opposite the first surface.

In an embodiment, the elastic laminate includes an inelastic zone in between the first stretch zone and the second stretch zone.

In an embodiment, the elastic laminate includes a third stretch zone having at least one stretch property different from the first stretch zone.

In an embodiment, the first stretch zone has a first level of extensibility, the second stretch zone has a second level of extensibility, and the third stretch zone has a third level of extensibility. In an embodiment, the third level of extensibility is different than the first level of extensibility and the second level of extensibility. In an embodiment, the third level of extensibility is the same as the first level of extensibility or the second level of extensibility.

In an embodiment, the elastic laminate includes a first inelastic zone between the first stretch zone and the second stretch zone, and a second inelastic zone between the second stretch zone and the third stretch zone.

According to an aspect of embodiments of the invention, there is provided an absorbent article that includes a chassis and the elastic laminate according to embodiments of the invention described herein attached to the chassis. In an embodiment, the elastic laminate is an ear. In an embodiment, the elastic laminate is a waist member. In an embodiment, the elastic laminate is a side panel. In an embodiment the elastic laminate is continuous around a circumference of the absorbent article.

These and other aspects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

The components of the following figures are illustrated to emphasize the general principles of the present disclosure and are not necessarily drawn to scale. Reference characters designating corresponding components are repeated as necessary throughout the figures for the sake of consistency and clarity.

FIG. 1 is a schematic illustration of an elastic laminate having an elastic film layer and a nonwoven layer attached to one side of the elastic film layer, according to embodiments of the invention;

FIG. 2 is a schematic illustration of an elastic laminate having an elastic film layer and a nonwoven layer attached to each side of the elastic film layer, according to embodiments of the invention;

FIG. 3 is a schematic illustration of an elastic laminate having two portions configured as tri-laminates and a single portion configured as a bi-laminate, according to embodiments of the invention;

FIG. 4A is a schematic illustration of an embodiment of the elastic film layer of FIGS. 1, 2 and 3;

FIG. 4B is a schematic illustration of an embodiment of the elastic film layer of FIGS. 1, 2 and 3;

FIG. 5A is a schematic illustration of an elastic laminate, in a relaxed state, having two stretch zones in a side-by-side configuration, according to embodiments of the invention;

FIG. 5B is a schematic illustration of the elastic laminate of FIG. 5A while the elastic laminate is in a stretched state;

FIG. 6A is a schematic illustration of an elastic laminate, in a relaxed state, having two stretch zones that are separated by an inelastic zone, according to embodiments of the invention;

FIG. 6B is a schematic illustration of the elastic laminate of FIG. 6A while the elastic laminate is in a stretched state;

FIG. 7A is a schematic illustration of an elastic laminate, in a relaxed state, having three stretch zones in a side-by-side configuration, according to embodiments of the invention;

FIG. 7B is a schematic illustration of the elastic laminate of FIG. 7A while the elastic laminate is in a stretched state;

FIG. 8 is a schematic illustration of an apparatus for manufacturing elastic laminate webs according to embodiments of the invention;

FIG. 9 is a schematic illustration of an activation station of the apparatus of FIG. 8, according to an embodiment of the invention;

FIG. 10 is a schematic illustration of the activation station, according to another embodiment of the invention;

FIG. 11 is a schematic illustration of the activation station, according to another embodiment of the invention;

FIG. 12 is a schematic illustration of an apparatus for manufacturing elastic laminate webs according to embodiments of the invention;

FIG. 13 is a schematic illustration of an apparatus for manufacturing elastic laminate webs according to embodiments of the invention;

FIG. 14 is a schematic illustration of an elastic laminate in accordance with an embodiment of the invention for incorporation into an absorbent article;

FIG. 15 is a schematic illustration of an elastic laminate in accordance with an embodiment of the invention for incorporation into an absorbent article;

FIG. 16 is a schematic illustration of an elastic laminate in accordance with an embodiment of the invention for incorporation into an absorbent article;

FIG. 17 is a schematic illustration of an absorbent article with the elastic laminate of FIG. 14 incorporated therein; and

FIG. 18 is a schematic illustration of an absorbent article with the elastic laminate of FIG. 16 incorporated therein.

### DETAILED DESCRIPTION

The term “web” as used herein refers to a material capable of being wound into a roll. Webs can be film webs, nonwoven webs, laminate webs, apertured laminate webs, etc. The face of a web refers to one of its two-dimensional surfaces, as opposed to its edge. The term “composite web” refers to a web that comprises two or more separate component webs that are attached to each other in a face to face relationship. Each of the separate component webs does not have to be continuous across the entire composite web and can have discontinuous parts. The attachment can be through the edges of the component webs, or the attachment can be at particular locations across the component webs, or the attachment can be continuous across the faces of the component webs.

The term “film” as used herein refers to a web made by extruding a molten sheet of thermoplastic polymeric material by a cast or blown extrusion process and then cooling said sheet to form a solid polymeric web. Films can be monolayer films, coextruded films, coated films, and composite films. Coated films are films comprising a monolayer or coextruded film that are subsequently coated (for example, extrusion coated, impression coated, printed, or the like) with a thin layer of the same or different material to which it is bonded. “Composite films” are films comprising layers of more than one component film and the component films are combined in a bonding process. Each of the separate component films does not have to be continuous across the entire composite film and can have discontinuous parts. Bonding processes may incorporate adhesive layers between the film layers.

The term “apertured film” as used herein denotes a film in which there exists a plurality of holes that extend from a first surface to a second surface, opposite the first surface. A two-dimensional apertured film is a film in which no three dimensional structure exists in the holes, which then connect the second surface of a flat film to the first surface of the film. A “formed film” is a three-dimensional film with protuberances, and a three-dimensional apertured film is a film in which a three-dimensional structure exists in the apertures (e.g., the apertures have a depth that is thicker than the thickness of the film) or the protuberances have apertures therethrough.

The term “nonwoven” as used herein means a material comprising a plurality of fibers. The fibers may be bonded to each other or may be unbonded. The fibers may be staple fibers or continuous fibers. The staple fibers may be thermal bonded carded fibers or air through bonded carded fibers. The continuous fibers may be meltblown fibers, spunlace fibers, spunbond fibers and the like, as well as combinations thereof. The fibers may comprise a single material or may comprise a multitude of materials, either as a combination of different fibers, or as a combination of similar fibers each comprised of different materials. As used herein, “nonwoven web” is used in its generic sense to define a nonwoven having a generally planar structure that is relatively flat, flexible and porous. The nonwoven web may be the product of any process for forming the same and may include a composite or combination of webs, such as, for example, a spunbond-meltblown-spunbond (“SMS”) nonwoven web.

The term “elastic” or “elastomeric” as used herein refers to a material having at least 80% recovery from 50% elongation. The term “inelastic” as used herein refers to a material that does not exhibit 80% recovery once elongated 50%. Inelastic materials may exhibit some level of elasticity but break or are permanently damaged when stretched beyond 50% elongation. As an example only, recovery testing may be performed by stretching a sample that is 1 inch wide with a gauge length of 2 inches to a “test elongation” at 20 inches/minute, held for 30 seconds, allowed to relax at 20 inches/minute to 0% extension, held for 60 seconds, and then stretched at 20 inches/minute. The “permanent set” is the elongation of the sample at which the load cell first detects a load in excess of 1 Newton on the second extension. The “percent recovery” is calculated as 100× (test elongation−permanent set)/test elongation. For example, when a length of material that was 10 inches in length in a normal resting state not under tension is elongated 50%, it is stretched by 5 inches to 15 inches in length. The material is then released and permitted to return to a resting state. If the length of the material at which the load cell first detects a load in excess of 1 Newton on the second extension is 11 inches or less, it is considered to have at least 80% recovery.

The term “stretch zone” or “elastic zone” as used herein refers to a portion of a web that is elastic when a force is applied to the web and released, and has a dimension of at least 3 mm wide in the direction of the force being applied to the web.

The term “dead zone” or “inelastic zone” as used herein refers to a portion of a web that is inelastic when a force is applied to the web and released. The material in a dead zone or inelastic zone may still show some level of elasticity, but as noted above will break or be permanently damaged when stretched beyond 50% elongation.

The term “extensibility” as used herein refers to the amount of elongation the material undergoes or the amount of strain the material incurs when subjected to a given load.

The term “stretch property” of a material as used herein includes any property related to the material's elastic characteristics and includes, without limitation, extensibility, modulus of elasticity (in tension, or Young's modulus), permanent set, etc.

The term “absorbent article” as used herein denotes articles that absorb and contain fluids and other exudates. Absorbent articles include garments that are placed against or in proximity to the body of a wearer to absorb and contain the various exudates discharged from a body. A non-exhaustive list of examples includes absorbent towels, diapers, training pants, absorbent underpants, adult incontinence products, feminine hygiene products and the like.

The term “activating” or “activation” as used herein refers to a process of stretching a material beyond a point where its physical properties are changed. In the case of a nonwoven web, sufficient activation of the web will result in the nonwoven web being more extensible and/or improving its tactile properties. In an activation process, forces are applied to a material causing the material to stretch. Formed film and nonwoven webs may be mechanically activated, for example. Mechanical activation processes comprise the use of a machine or apparatus to apply forces to the web to cause stretching of the web. Methods and apparatus used for activating webs of materials include, but are not limited to, activating the web through intermeshing gears or plates, activating the web through incremental stretching, activating the web by ring rolling, activating the web by tenter frame stretching, canted wheel stretchers or bow rollers, and activating the web in the machine direction between nips or roll stacks operating at different speeds to mechanically stretch the components, and combinations thereof.

Various embodiments of the present invention will now be highlighted. The discussion of any one embodiment is not intended to limit the scope of the present invention. To the contrary, aspects of the embodiments are intended to emphasize the breadth of the invention, whether encompassed by the claims or not. Furthermore, any and all variations of the embodiments, now known or developed in the future, also are intended to fall within the scope of the invention.

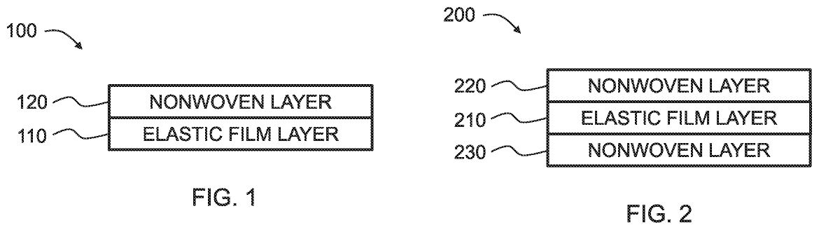

FIG. 1 schematically illustrates an elastic laminate 100 in accordance with embodiments of the invention. As illustrated, the elastic laminate 100 is a so-called “bi-laminate” having an elastic film layer 110 attached to a nonwoven layer 120 on one side thereof. The elastic film layer 110 may be continuous across the entire elastic laminate 100, or may be discontinuous in one or more directions and located in sections or strips of the elastic laminate 100. The nonwoven layer 120 may be continuous across the entire elastic laminate 100 or may be discontinuous in one or more direction and located in sections or strips of the elastic laminate 100. Additional aspects of embodiments of the elastic laminate 100 will be described in further detail below.

FIG. 2 schematically illustrates an elastic laminate 200 in accordance with embodiments of the invention. As illustrated, the elastic laminate 200 is a so-called “tri-laminate” having an elastic film layer 210, a first nonwoven layer 220 on one side of the elastic film layer 210, and a second nonwoven layer 230 on an opposite side of the elastic film layer 210 as the first nonwoven layer 220. Additional aspects of embodiments of the elastic laminate 200 will be described in further detail below.

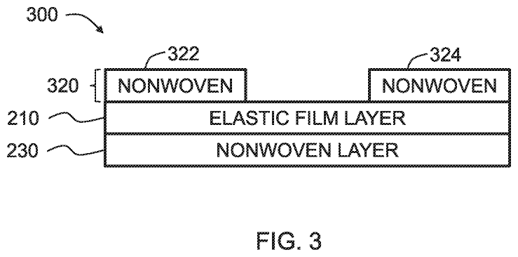

FIG. 3 schematically illustrates an elastic laminate 300 in accordance with embodiments of the invention. As illustrated, the elastic laminate 300 is similar to the tri-laminate elastic laminate web 200 illustrated in FIG. 2 and has the elastic film layer 210 and the nonwoven layer 230 on one side of the elastic film layer 210, but instead of having the continuous nonwoven layer 220 on the opposite side of the elastic film layer 210 as the nonwoven layer 230, the elastic laminate 300 has a nonwoven layer 320 that includes separate sections of nonwoven material 322, 324. Such a configuration provides a tri-laminate at the locations of the sections of nonwoven material 322, 324 and a bi-laminate in between the locations of the sections of nonwoven material 322, 324. The illustrated embodiments of the elastic laminate 100, 200, 300 are not intended to be limiting in any way, and other configurations of an elastic laminate web are contemplated as being with the scope of embodiments of the inventions. For example, in an embodiment, the elastic film layer 110, 210 may be discontinuous and include separate sections of elastic film across the nonwoven layer(s) 120, 220, 230, 320.

Each nonwoven layer 120, 220, 230, 320 may be made from any suitable nonwoven material that includes fibrous materials, such as staple fiber materials including thermal bonded carded fibers and air through bonded carded fibers, continuous fiber materials including meltblown fibers, spunlace fibers, spunbond fibers, and the like, as well as combinations thereof. In an embodiment, the nonwoven material may have a spunbond-meltblown-spunbond (“SMS”) construction or a spunbond-meltblown-meltblown-spunbond (“SMMS”) construction. The fibers within the nonwoven material may be made of polyethylene (PE), polypropylene (PP), bicomponent or blends of PE and PP, or other materials, such as polyethylene terephthalate (PET). In an embodiment, the fibers may include natural fibers, such as cotton and/or cellulose. Additionally, the nonwoven material may be homogeneous or contain a variety of materials including bicomponent fibers (e.g. having an inner core of one material and an outer core of a second material), and fibers of different morphologies, geometries, and surface finishes. The basis weight of the nonwoven material may be in the range of about 8 grams per square meter (“gsm”) to about 100 gsm.

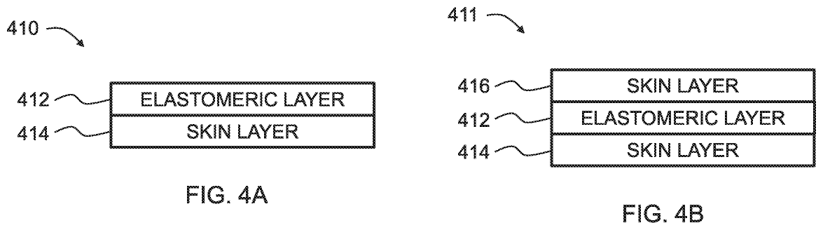

FIG. 4A schematically illustrates an embodiment of an elastic film layer 410 that may be used as the elastic film layers 110, 210, 310 of the elastic film laminates 100, 200, 300 of FIGS. 1-3. The elastic film layer 410 includes an elastomeric material layer 412 and a first skin layer 414 on one side thereof. In an embodiment, the elastic film layer 410 may also include a second skin layer on an opposite side of the elastomeric material layer 412 as the skin layer 414. For example, FIG. 4B schematically illustrates an embodiment of an elastic film layer 411 that may be used as the elastic film layers 110, 210, 310 and includes the elastomeric material layer 412, the first skin layer 414 on one side of the elastomeric material layer 412, and a second skin layer 416 on an opposite side of the elastomeric material layer 412 as the first skin layer 414. The illustrated embodiments are not intended to be limiting in any way. For example, in an embodiment, the elastic film layer 410 may not have a skin layer and may only be made from the elastomeric layer 412. In an embodiment, additional layers may be used to make the elastic film layer 410, such as additional elastomeric layers and/or additional skin layers and/or additional layers in between the elastomeric material layer 412 and the skin layers 414, 416. In an embodiment, the elastic film layer 410, 411 may be an apertured film and include a plurality of two-dimensional apertures, or a formed film and include a plurality of three-dimensional protuberances, or a three-dimensional apertured film.

The elastomeric material layer 412 may be made from any suitable elastic material, such as natural or synthetic polymeric materials. Examples of suitable polymeric materials include low crystallinity polyethylene, metallocene catalyzed low crystallinity polyethylene, polyolefin based elastomers such as INFUSE™ olefin block copolymers manufactured by Dow Chemical Company, VISTAMAXX™ performance polymers manufactured by Exxon Mobil Corporation, and the like, ethylene vinyl acetate copolymers (“EVA”), polyurethane, polyisoprene, polyurethane, polyisoprene, butadiene-styrene copolymers, styrene block copolymers such as styrene/isoprene/styrene (“SIS”), styrene/butadiene/styrene (“SBS”), styrene/ethylene-butadiene/styrene (“SEBS”), or styrene/ethylene-propylene/styrene (“SEPS”) block copolymers. Blends of these polymers alone or with other modifying elastic or non-elastomeric materials may also be used. For example, the elastomeric material layer 122, 222 may be made from blends of styrene block copolymers with polyolefins, such as polyethylene or polypropylene, polyolefin-based elastomers, and/or any combination thereof, or any other suitable elastic material.

Each skin layer 414, 416 may include a suitable material that is more or less elastic than the elastomeric material layer 412. In an embodiment, each skin layer 414, 416 may include one or more polyolefins, such as polyethylene or polypropylene.

The thickness of the elastic film layer 410, 411 may be in the range of about 10 microns to about 200 microns. The basis weight of the elastic film layer 410, 411 may be in the range of about 10 grams per square meter (“gsm”) to about 200 gsm. The elastomeric layer 412 within the elastic film layer 410, 411 may have a thickness in the range of about 10 microns to about 200 microns, and each of the skin layers 414, 416 may have a thickness in the range of about 1 micron to 50 microns.

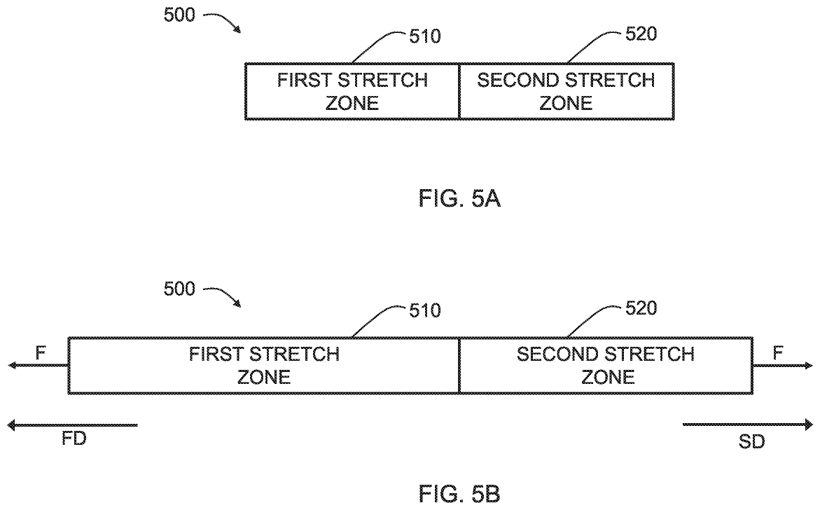

FIG. 5A is a schematic illustration of an elastic laminate 500, which may be any of the elastic laminates 100, 200, 300 described above, having a first stretch zone 510 and a second stretch zone 520 in a side-by-side configuration. The first stretch zone 510 exhibits at least one stretch property that is different from the second stretch zone 520 when a force F is applied to the elastic laminate 500 in a first direction FD and/or a second direction SD opposite the first direction FD, as illustrated in FIG. 5B. If one end of the first stretch zone 510 or the second stretch zone 520 is anchored, the force F may be applied to the unanchored end. As schematically illustrated in FIGS. 5A and 5B, even though the first stretch zone 510 and the second stretch zone 520 have the same initial size, when a force is applied to each end of the elastic laminate 500 in the first direction FD and the second direction SD, the material in the first stretch zone 510 stretches (elongates) further than the material in the second stretch zone 520, indicating that the material in the first stretch zone 510 exhibits a greater level of extensibility than the material in the second stretch zone 520. The material in the first stretch zone 510 may also have a lower modulus of elasticity (Young's modulus) and exhibit a lower or higher permanent set than the material in the second stretch zone 520.

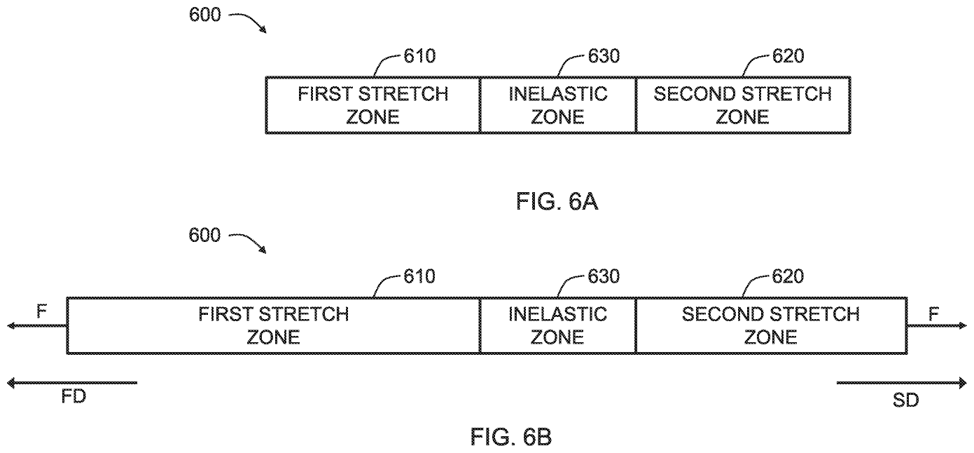

FIG. 6A is a schematic illustration of an elastic laminate 600, which may be any of the elastic laminates 100, 200, 300 described above, having a first stretch zone 610 and a second stretch zone 620 that are separated by an inelastic zone 630. The first stretch zone 610, the second stretch zone 620, and the inelastic zone 630 each exhibit at least one stretch property that is different from the other zones when a force F is applied to the elastic laminate 600 in the first direction FD and/or the second direction SD, as illustrated in FIG. 6B. As schematically illustrated in FIGS. 6A and 6B, even though the first stretch zone 610 and the second stretch zone 620 have the same initial size, when a force is applied to each end of the elastic laminate 600 in the first direction FD and the second direction SD, the material in the first stretch zone 610 stretches (elongates) further than the material in the second stretch zone 620, indicating that the material in the first stretch zone 610 exhibits a greater level of extensibility than the material in the second stretch zone 620. The material in the first stretch zone 610 may also have a lower modulus of elasticity (Young's modulus) and exhibit a lower or higher permanent set than the material in the second stretch zone 620. In contrast, the inelastic zone 630 does not exhibit any appreciable elongation, thereby indicating the material within the inelastic zone 630 has a lower level of extensibility, higher modulus of elasticity and/or higher permanent set.

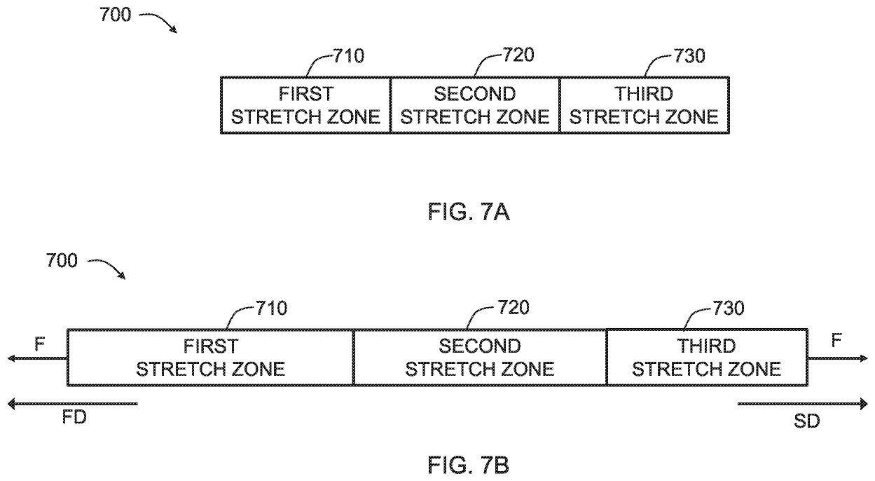

FIG. 7A is a schematic illustration of an elastic laminate 700, which may be any of the elastic laminates 100, 200, 300 described above, having a first stretch zone 710, a second stretch zone 720, and a third stretch zone 730 next to the second stretch zone 720. Each of the three stretch zones 710, 720, 730 has its own stretch properties that may be different than at least one of the stretch properties of one or more of the other stretch zones. For example, the first stretch zone 710 may exhibit a first level of extensibility, the second stretch zone 720 may exhibit a second level of extensibility, different from the first level of extensibility, and the third stretch zone may exhibit a third level of extensibility, different from at least one of the first and second levels of extensibility, upon stretching the elastic laminate 700 in the first direction FD and/or the second direction SD, as indicated by the arrows FD, SD in FIG. 7B. In the embodiment illustrated in FIG. 7B, the material in the first stretch zone 710 is more elastic (lower modulus of elasticity) and has a greater level of extensibility than the material in the second stretch zone 720, and the material in the second stretch zone 720 is more elastic (lower modulus of elasticity) and has a greater level of extensibility than the material in the third stretch zone 730.

Additional stretch zones and/or inelastic zones may be used across the elastic laminate 500, 600, 700. The illustrated embodiments are not intended to be limiting in any way. For example, inelastic zones may be added in between the first stretch zone 710 and the second stretch zone 720, as well as in between the second stretch zone 720 and the third stretch zone 730 of the elastic laminate 700 of FIG. 7A, or at one or more ends of the elastic laminates 500, 600, 700 of FIGS. 5A, 6A and 7A.

In an embodiment, the second level of extensibility in the second stretch zone 520, 620, 720 may be in the range of about 10% to about 90% of the first level of extensibility in the first stretch zone 510, 610, 710. In an embodiment, the second level of extensibility may be in the range of about 20% to about 80% of the first level of extensibility. In an embodiment, the second level of extensibility may be in the range of about 30% to about 70% of the first level of extensibility. Similarly, the third level of extensibility in the third stretch zone 730 may be in the range of about 10% to about 90% of the first level of extensibility in the first stretch zone 710. In an embodiment, the third level of extensibility may be in the range of about 20% to about 80% of the first level of extensibility. In an embodiment, the third level of extensibility may be in the range of about 30% to about 70% of the first level of extensibility.

In an embodiment, the second stretch zone 520, 620, 720 may have a second modulus of elasticity in the range of about 10% to about 90% of a first modulus of elasticity of the first stretch zone 510, 610, 710. In an embodiment the second modulus of elasticity may be in the range of about 20% to about 80% of the first modulus of elasticity. In an embodiment the second modulus of elasticity may be in the range of about 30% to about 70% of the first modulus of elasticity. Similarly, the third stretch zone 730 may have a third modulus of elasticity in the range of about 10% to about 90% of the first modulus of elasticity of the first stretch zone 710. In an embodiment, the third modulus of elasticity may be in the range of about 20% to about 80% of the first modulus of elasticity. In an embodiment, the third modulus of elasticity may be in the range of about 30% to about 70% of the first modulus of elasticity.

In an embodiment, the second stretch zone 520, 620, 720 may have a second permanent set in the range of about 50% to about 150% of a first permanent set of the first stretch zone 510, 610, 710. In an embodiment, the second permanent set may be in the range of about 75% to about 125% of the first permanent set. Similarly, in an embodiment, the third stretch zone 730 may have a third permanent set in the range of about 50% to about 150% of the first permanent set of the first stretch zone 710. In an embodiment, the third permanent set may be in the range of about 75% to about 125% of the first permanent set.

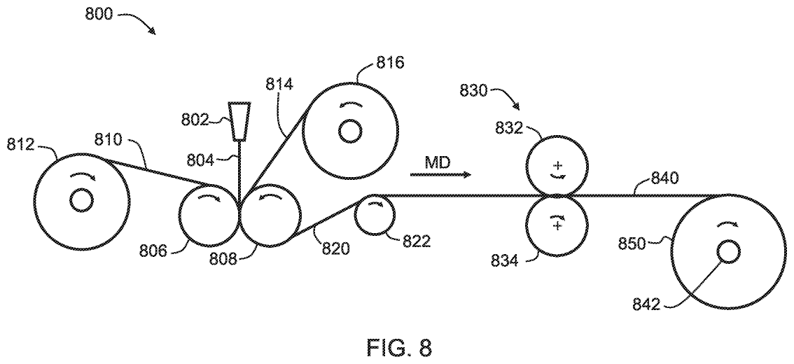

FIG. 8 is a schematic illustration of an apparatus 800 for manufacturing elastic laminates, such as the elastic laminates 100, 200, 300, 500, 600, 700 according to embodiments of the invention. As illustrated, the apparatus 800 includes an extrusion die 802 constructed and arranged to extrude a polymer melt curtain (molten polymer web) 804 between a first roller 806 and a second roller 808 (in proximity to each other). Also fed between the first roller 806 and the second roller 808 are a first nonwoven web 810, unwound from a first nonwoven supply roll 812, and a second nonwoven web 814, unwound from a second nonwoven supply roll 816. In proximity to the first roller 806 and the second roller 808, the fibers of the nonwoven webs 810, 814 may embed partially into the molten polymer web 804 to create an elastic laminate precursor material 820. In an embodiment, only one of the nonwoven webs 810 or 814 may be fed between the first roller 806 and the second roller 808 to form a two-layer elastic laminate precursor material.

The illustrated embodiment is not intended to be limiting in any way. For example, in an embodiment, an already extruded film web having an elastomeric material layer may be reheated and fed between the first roller 806 and the second roller 808. Such an already extruded film web may be solid or may be apertured or may be a formed film. In an embodiment, an adhesive may be provided to an elastic film web and/or one or both of the nonwoven webs 810, 814 prior to the webs being fed between the first roller 806 and the second roller 808. Any lamination technique may be used to attach the layers of the elastic laminate webs to create the elastic laminate precursor material 820, as would be understood by one of ordinary skill in the art.

After the elastic laminate precursor material 820 is created, a third roller 822 may be used to transport the elastic laminate precursor material 820 in the machine direction MD to an activation station 830 that includes a first intermeshing gear (“IMG”) roller 832 and a second intermeshing gear (“IMG”) roller 834. Additional rollers may be used to convey the elastic laminate precursor material 820 in the machine direction MD. The illustrated embodiment is not intended to be limiting in any way.

As discussed in further detail below, the first IMG roller 832 and the second IMG roller 834 are designed to create multiple (i.e., at least two) stretch zones in the elastic laminate precursor material 820 in the transverse direction (TD) to form an elastic laminate 840 according to embodiments of the invention. After the multiple stretch zones are created, the elastic laminate 840 may be wound about a spindle 842 into a roll 850.

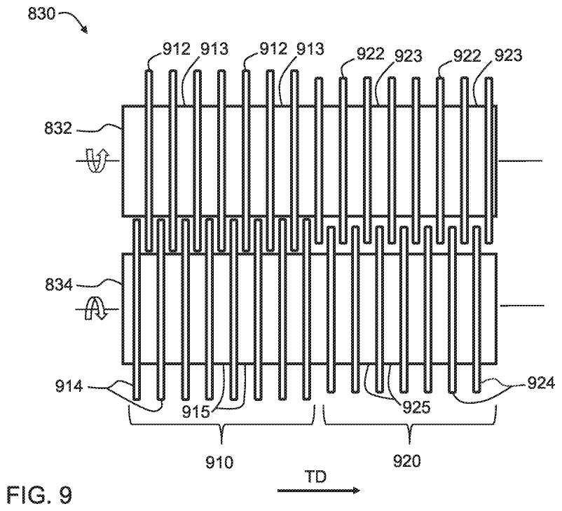

FIG. 9 schematically illustrates an embodiment of the first IMG roller 832 and the second IMG roller 834 that may be used in the activation station 830 of the apparatus 800 of FIG. 8 for TD activation. As illustrated, the IMG rollers 832, 834 have their axes of rotation disposed in parallel relationship. The first IMG roller 832 includes a first plurality of axially-spaced, side-by-side, circumferentially-extending, equally-configured gears 912 that can be in the form of thin fins having a generally rectangular cross section, as well as a second plurality of axially-spaced, side-by-side, circumferentially-extending, equally-configured gears 922 that can be in the form of thin fins having a generally rectangular cross section. The second IMG roller 834 includes a first plurality of axially-spaced, side-by-side, circumferentially-extending, equally-configured gears 914 that can be in the form of thin fins having a generally rectangular cross section, as well as a second plurality of axially-spaced, side-by-side, circumferentially-extending, equally-configured gears 924 that can be in the form of thin fins having a generally rectangular cross section.

The first plurality of gears 912 of the first IMG roller 832 complement the first plurality of gears 914 of the second IMG roller 834 in a first zone 910 that extends in the transverse direction TD, and the second plurality of gears 922 of the first IMG roller 832 complement the second plurality of gears 924 of the second IMG roller 834 in a second zone 920 that is adjacent to the first zone 910 and extends in the transverse direction TD.

The spaces between adjacent gears 912, 922, 914, 924 define recessed, circumferentially-extending, equally configured grooves 913, 923, 915, 925, respectively. The grooves 913, 923, 915, 925 may have a generally rectangular cross section when the gears 912, 922, 914, 924 have a generally rectangular cross section. Desirably, the grooves 913, 915 have a larger width than that of the gears 912, 914 to permit the material that passes between the IMG rollers 832, 834 to be received within the respective grooves 913, 915 and locally stretched in the first zone 910. Similarly, the grooves 923, 925 desirably have a larger width than that of the gears 922, 924 to permit the material that passes between the IMG rollers 832, 834 to be received within the respective grooves 923, 925 and locally stretched in the second zone 920.

The spacing and the depth of engagement of the gears 912, 914 and 922, 924 within a respective zone 910, 920 determines the degree of elongation to which the elastic laminate precursor material 820 is subjected. In the embodiment illustrated in FIG. 9, all of the gears 912, 914, 922, 924 have the same spacing, but the gears 912, 914 of the first zone 910 have a greater depth of engagement and are therefore configured to stretch and elongate (i.e., activate) the elastic laminate precursor material 820 to a greater degree than the gears 922, 924 of the second zone 920.

The configuration illustrated in FIG. 9 results in the elastic laminate 840 having the same configuration as the elastic laminate 500 of FIGS. 5A and 5B, with the first stretch zone 510 created in the first zone 910 of the activation station 830 having at least one stretch property that is different from at least one stretch property of the second stretch zone 520 created in the second zone 920 of the activation station 830 when the elastic laminate 500 is subjected to the force F in the first direction FD and/or the second direction SD.

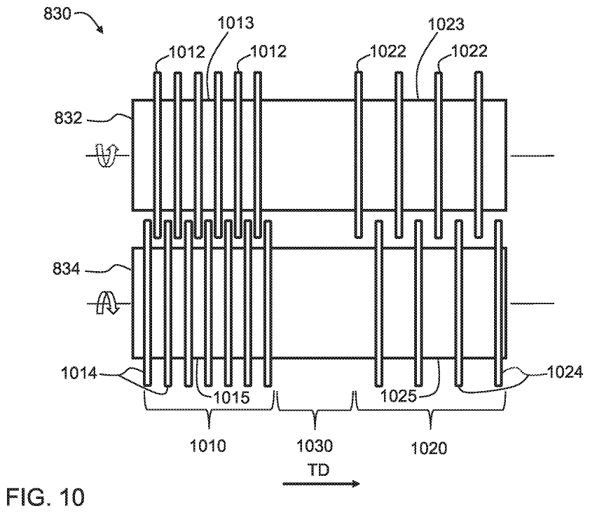

FIG. 10 schematically illustrates another embodiment of the first IMG roller 832 and the second IMG roller 834 that may be used in the activation station 830 of the apparatus 800 of FIG. 8 for TD activation. The first IMG roller 832 includes a first plurality of axially-spaced, side-by-side, circumferentially-extending, equally-configured gears 1012 that can be in the form of thin fins having a generally rectangular cross section, as well as a second plurality of axially-spaced, side-by-side, circumferentially-extending, equally-configured gears 1022 that can be in the form of thin fins having a generally rectangular cross section. The second IMG roller 834 includes a first plurality of axially-spaced, side-by-side, circumferentially-extending, equally-configured gears 1014 that can be in the form of thin fins having a generally rectangular cross section, as well as a second plurality of axially-spaced, side-by-side, circumferentially-extending, equally-configured gears 1024 that can be in the form of thin fins having a generally rectangular cross section.

The first plurality of gears 1012 of the first IMG roller 832 complement the first plurality of gears 1014 of the second IMG roller 834 in a first zone 1010 that extends in the transverse direction TD, and the second plurality of gears 1022 of the first IMG roller 832 complement the second plurality of gears 1024 of the second IMG roller 834 in a second zone 1020 that is adjacent to the first zone 1010 and extends in the transverse direction TD. A third zone 1030, which does not include any gears is located between the first zone 1010 and the second zone 1020.

The spaces between adjacent gears 1012, 1022, 1014, 1024 define recessed, circumferentially-extending, equally configured grooves 1013, 1023, 1015, 1025, respectively. The grooves 1013, 1023, 1015, 1025 may have a generally rectangular cross section when the gears 1012, 1022, 1014, 1024 have a generally rectangular cross section. The gears 1012, 1014 and the grooves 1013, 1015 of the first zone 1010 need not each be of the same width and desirably, the grooves 1013, 1015 have a larger width than that of the gears 1012, 1014 to permit the material that passes between the IMG rollers 832, 834 to be received within the respective grooves 1013, 1015 and locally stretched in the first zone 1010.

As illustrated in FIG. 10, the grooves 1023, 1025 of the second zone 1020 have a much greater width than the respective gears 1022, 1024 of the second zone, and also than the grooves 1013, 1015 of the first zone 1010, while the depth of engagement of all of the gears 1012, 1014, 1022, 1024 have the same depth of engagement. The greater spacing between the gears 1022, 1024 provides less stretch and elongation of the elastic laminate precursor material 820 passing through the second zone 1020, and therefore a lower level of extensibility than the level of extensibility imparted to the elastic laminate precursor material 820 that passes through the first zone 1010.

The configuration illustrated in FIG. 10 results in the elastic laminate 840 having the same configuration as the elastic laminate 600 of FIGS. 6A and 6B, with the first stretch zone 610 created in the first zone 1010 of the activation station 830 having at least one stretch property that is different from at least one stretch property of the second stretch zone 620 created in the second zone 1020 of the activation station 830. Because the third zone 1030 does not include gears, the center of the elastic laminate precursor material 820 is not stretched or elongated, thereby allowing for the inelastic zone 630 between the first stretch zone 610 and the second stretch zone 620.

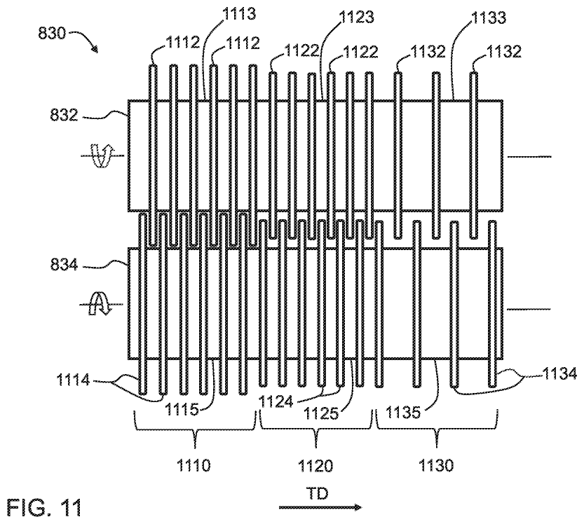

FIG. 11 schematically illustrates another embodiment of the first IMG roller 832 and the second IMG roller 834 that may be used in the activation station 830 of the apparatus 800 of FIG. 8 for TD activation. The first IMG roller 832 includes a first plurality of axially-spaced, side-by-side, circumferentially-extending, equally-configured gears 1112 that can be in the form of thin fins having a generally rectangular cross section, a second plurality of axially-spaced, side-by-side, circumferentially-extending, equally-configured gears 1122 that can be in the form of thin fins having a generally rectangular cross section, and a third plurality of axially-spaced, side-by-side, circumferentially-extending, equally-configured gears 1132 that can be in the form of thin fins having a generally rectangular cross section. The second IMG roller 834 includes a first plurality of axially-spaced, side-by-side, circumferentially-extending, equally-configured gears 1114 that can be in the form of thin fins having a generally rectangular cross section, a second plurality of axially-spaced, side-by-side, circumferentially-extending, equally-configured gears 1124 that can be in the form of thin fins having a generally rectangular cross section, and a third plurality of axially-spaced, side-by-side, circumferentially-extending, equally-configured gears 1134 that can be in the form of thin fins having a generally rectangular cross section.

The first plurality of gears 1112 of the first IMG roller 832 complement the first plurality of gears 1114 of the second IMG roller 834 in a first zone 1110 that extends in the transverse direction TD, the second plurality of gears 1122 of the first IMG roller 832 complement the second plurality of gears 1124 of the second IMG roller 834 in a second zone 1120 that is adjacent to the first zone 1110 and extends in the transverse direction TD, and the third plurality of gears 1132 of the first IMG roller 832 complement the third plurality of gears 1134 of the second IMG roller 834 in a third zone 1130 that is adjacent to the second zone 1120 and extends in the transverse direction TD.

The spaces between adjacent gears 1112, 1122, 1132, 1114, 1124, 1134 define recessed, circumferentially-extending, equally configured grooves 1113, 1123, 1133, 1115, 1125, 1135, respectively. The grooves 1113, 1123, 1133, 1115, 1125, 1135 may have a generally rectangular cross section when the gears 1112, 1122, 1132, 1114, 1124, 1134 have a generally rectangular cross section. Desirably, the grooves 1113, 1115 have a larger width than that of the gears 1112, 1114 to permit the material that passes between the IMG rollers 832, 834 to be received within the respective grooves 1113, 1115 and locally stretched in the first zone 1110.

In the embodiment illustrated in FIG. 11, all of the gears 1112, 1114, 1122, 1124 in the first zone 1110 and the second zone 1120 have the same spacing, but the gears 1112, 1114 of the first zone 1110 have a greater depth of engagement and are configured to stretch and elongate (i.e., activate) the elastic laminate precursor material 820 to a greater degree than the gears 1122, 1124 of the second zone 1120, similar to the configuration of the first zone 910 and the second zone 920 illustrated in FIG. 9.

As illustrated in FIG. 11, the grooves 1133, 1135 of the third zone 1130 have a much greater width than the respective gears 1132, 1134 of the third zone 1130, and also than the grooves 1123, 1125 of the second zone 1120, while the depth of engagement of all of the gears 1122, 1124, 1132, 1134 of the second zone 1120 and the third zone 1130 have the same depth of engagement. The greater spacing between the gears 1132, 1134 provides less stretch and elongation of the elastic laminate precursor material 820 passing through the third zone 1130, and therefore a lower level of extensibility than the level of extensibility imparted to the elastic laminate precursor material 820 that passes through the first zone 1110 and the second zone 1120.

The configuration illustrated in FIG. 11 results in the elastic laminate 840 having the same configuration as the elastic laminate 700 of FIGS. 7A and 7B, with the first stretch zone 710 created in the first zone 1110 of the activation station 830 having at least one stretch property different from at least one stretch property of the second stretch zone 720 created in the second zone 1120 of the activation station 830, and the second stretch zone 720 having at least one stretch property different from at least one stretch property of the third stretch zone 730 created in the third zone 1130 of the activation station 830.

Other embodiments of the first IMG roller 832 and the second IMG roller 834 may be used in the activation station 830 of the apparatus 800 of FIG. 8 for TD activation to achieve the desired level of activation and stretch properties in the desired number of zones. The illustrated embodiments are not intended to be limiting in any way. In an embodiment, the first IMG roller 832 and the second IMG roller 834 may have corresponding gears and grooves that are designed to provide a gradient in the transverse direction TD. For example the gears of the first IMG roller 832 and the second IMG roller 834 may have a depth of engagement that is deepest at one end of the first IMG roller 832 and the second IMG roller 834 and shallowest at the opposite end of the first IMG roller 832 and the second IMG roller 834 in the transverse direction TD, with the depths of engagement of the gears in between the ends gradually decreasing from the deepest to the shallowest depths of engagement. Such an arrangement will generate an elastic laminate that has different stretch properties across its width in the transverse direction TD. A similar effect may be created by changing the spacing between the gears of the first IMG roller 832 and the second IMG roller 834, as would be understood by one of skill in the art.

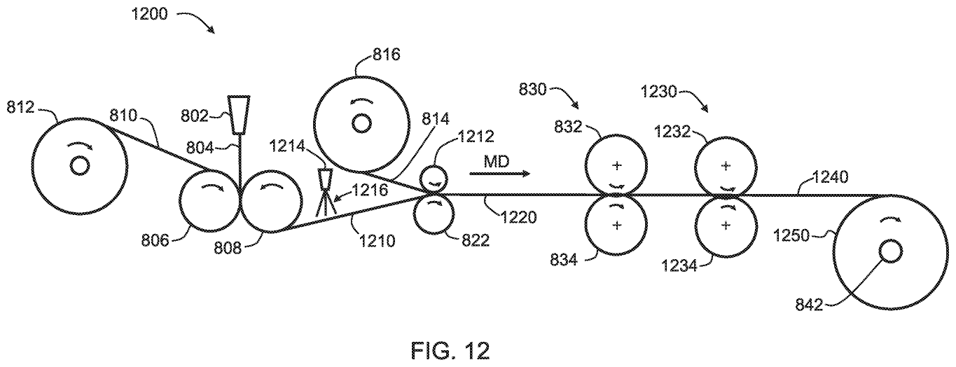

FIG. 12 is a schematic illustration of an apparatus 1200 for manufacturing elastic laminates 100, 200, 300, 500, 600, 700 according to embodiments of the invention. The apparatus 1200 has many of the same parts as the apparatus 800 illustrated in FIG. 8 with a few notable differences. For example, the apparatus 1200 includes the extrusion die 802 constructed and arranged to extrude the polymer melt curtain 804 between the first roller 806 and the second roller 808 while the first nonwoven web 810 is also fed in between the first roller 806 and the second roller 808 to form a film nonwoven bi-laminate web 1210. The second nonwoven web 814 is fed in between the third roller 822 and a fourth roller 1212. An adhesive applicator 1214 provides an adhesive 1216 to the film/nonwoven bi-laminate web 1210 prior to the film/non-woven bi-laminate web 1210 being fed between the third roller 822 and the fourth roller 1212. In an embodiment, the adhesive 1216 may be applied to the second nonwoven web 814. The illustrated embodiment is not intended to be limiting in any way. The third roller 822 and the fourth roller 1212 apply a suitable amount of pressure to bond the second nonwoven web 814 to the film/nonwoven laminate 1210 to form an elastic laminate precursor 1220.

After the elastic laminate precursor material 1220 is created, the elastic laminate precursor material 1220 is conveyed in the machine direction MD to the activation station 830 that includes the first IMG roller 832 and the second IMG roller 834, and then to an optional second activation station 1230 that also includes a first IMG roller 1232 and a second IMG roller 1234. The combination of the two activation stations 830, 1230 may be used to create the desired multiple stretch zones in the elastic laminate precursor material 1220 in the transverse direction TD to form an elastic laminate 1240 according to embodiments of the invention. For example, a first level of stretch properties may be created across at least a portion of the elastic laminate precursor material 1220 in the transverse direction at the first activation station 830, and one or more zones may be used to increase at least one stretch property, such as extensibility, to a second level for only a portion (or portions) of the elastic laminate precursor material 1220 at the second activation station 1230 to create multiple stretch zones. Other configurations of the IMG rollers 832, 834, 1232, 1234 may be used to create the desired stretch properties across the elastic laminate 1240, as would be understood by one of ordinary skill in the art. After the multiple stretch zones are created, the elastic laminate 1240 may be wound about the spindle 842 into a roll 1250.

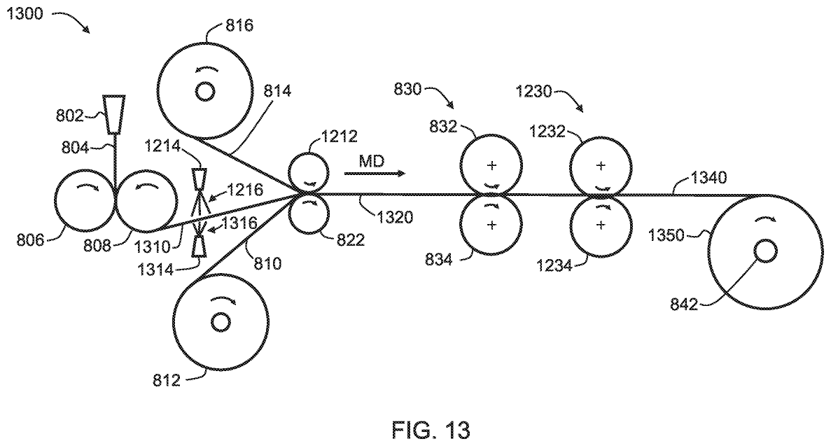

FIG. 13 is a schematic illustration of an apparatus 1300 for manufacturing elastic laminates according to embodiments of the invention. The apparatus 1300 has many of the same parts as the apparatus 1200 illustrated in FIG. 12 with a few notable differences. For example, the apparatus 1300 includes the extrusion die 802 constructed and arranged to extrude the polymer melt curtain 804 between the first roller 806 and the second roller 808 to create a solid film web 1310. The adhesive applicator 1214 provides the adhesive 1216 to one side of the solid film web 1310, and a second adhesive applicator 1314 provides an adhesive 1316 to the other side of the solid film web 1310 before the solid film web 1310 travels between the third roller 822 and the fourth roller 1212. The first nonwoven web 810 and the second nonwoven web 814 are also fed between the third roller 822 and the fourth roller 1212. In an embodiment, the adhesive 1316 may be applied to the first nonwoven web 810 and/or the adhesive 1216 may be applied to the second nonwoven web 814 before being fed between the third roller 822 and the fourth roller 1212. The illustrated embodiment is not intended to be limiting in any way. The third roller 822 and the fourth roller 1212 apply a suitable amount of pressure to bond the first nonwoven web 810 and the second nonwoven web 814 to opposite sides of the sold film web 1310 to form an elastic laminate precursor 1320.

After the elastic laminate precursor material 1320 is created, the elastic laminate precursor material 1320 is conveyed in the machine direction MD to the activation station 830 and then to the optional second activation station 1230 to create the desired multiple stretch zones in the elastic laminate precursor material 1320 in the transverse direction TD to form an elastic laminate 1340 according to embodiments of the invention. After the multiple stretch zones are created, the elastic laminate 1340 may be wound about the spindle 842 into a roll 1350.

In an embodiment, one or both of the activation stations 830, 1230 may be configured to provide machine direction (MD) activation. In MD activation, a view of the cross section of the IMG rollers 832, 834, 1232, 1234 looking down the axes of the rotatable shafts of the IMG rollers 832, 834, 1232, 1234 would show gear teeth (not shown) cut into and around the circumference of the IMG rollers 832, 834, 1232, 1234 with their long axes substantially parallel with the axes of the IMG rollers 832, 834, 1232, 1234. The teeth on one IMG roller 832, 1232 meshes into the grooves on the adjacent IMG roller 834, 1234 in order to provide a stretching action to the elastic laminate precursor material 1320 in the machine direction MD. The depth of engagement of the gear teeth and/or spacing of the gear teeth may be varied around the circumference of the IMG rollers 832, 834, 1232, 1234 to create multiple stretch zones having a least one different stretch property in the machine direction MD in a similar manner described above with respect to TD activation.

Other methods and apparatus may be used to create different levels of stretch properties for different stretch zones by using different activation techniques known in the art. For example a so-called stretch and bond process in which the elastic film layer and/or the nonwoven web(s) are stretched and then bonded together while in an extended state may be used to create different stretch zones. In an embodiment, a zoned extrusion die may be used as the extrusion die 802 to create a polymer melt curtain 804 having different zones of materials with different stretch properties so that when the elastic laminate precursor material 820, 1220, 1320 enters the activation station 830 having IMG rollers 832, 834 with uniform complementary gears and grooves, the resulting elastic laminate 840, 1240, 1340 will have different stretch zones exhibiting different stretch properties, such as different levels of extensibility in accordance with the different stretch zones of materials.

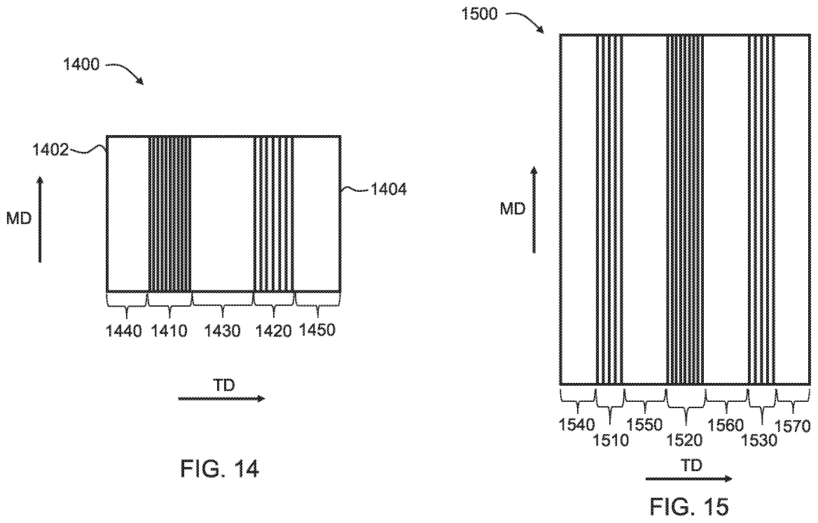

FIG. 14 is a schematic illustration of an elastic laminate 1400 in accordance with an embodiment of the invention. The elastic laminate 1400 may be used as a portion of an absorbent article. More specifically, the elastic laminate 1400 may be an ear or flap of a diaper. The elastic laminate 1400 has a proximal end 1402 configured to be attached to a chassis of, for example, a diaper, and a distal end 1404 configured to be attached to a fastener tab, such as a hook part of a hook and loop type fastener. The elastic laminate 1400 includes a first stretch zone 1410 and a second stretch zone 1420 that is spaced in the transverse direction TD from the first stretch zone 1410 by a first inelastic zone 1430. A second inelastic zone 1440 is located proximal to the first stretch zone 1410 and defines the proximal end 1402 of the elastic laminate 1400. A third inelastic zone 1450 is located distal of the second stretch zone 1420 and defines the distal end 1404 of the elastic laminate 1400. The first stretch zone 1410 and the second stretch zone 1420 have at least one different stretch property, such as different levels of extensibility, when a force is applied to the distal end 1404 and the elastic laminate 1400 is stretched in the transverse direction TD.

FIG. 15 is a schematic illustration of an elastic laminate 1500 in accordance with an embodiment of the invention. The elastic laminate 1500 includes a first stretch zone 1510, a second stretch zone 1520, and a third stretch zone 1530. In an embodiment, the three stretch zones 1510, 1520, 1530 may each have at least one different stretch property, such as a different level of extensibility, when the elastic laminate 1500 is stretched in the transverse direction TD. In an embodiment, two of the three stretch zones, such as the first stretch zone 1510 and the third stretch zone 1530, may have the same level of extensibility, while the remaining stretch zone, such as the second stretch zone 1520, may have a different level of extensibility than the other two stretch zones 1510, 1530. In an embodiment, the second stretch zone 1520 may have a higher level of extensibility, for example, than the other two stretch zones 1510, 1530. In an embodiment, the second stretch zone 1520 may have a lower level of extensibility, for example, than the other two stretch zones 1510, 1530.

As illustrated in FIG. 15, a first inelastic zone 1540 may be positioned at one edge of the elastic laminate 1500, a second inelastic zone 1550 may be positioned between the first stretch zone 1510 and the second stretch zone 1520, a third inelastic zone 1560 may be positioned between the second stretch zone 1520 and the third stretch zone 1530, and a fourth inelastic zone 1570 may be positioned at the other edge of the elastic laminate 1500. More or less stretch zones and/or inelastic zones may be created in the elastic laminate 1500. The illustrated embodiment is not intended to be limiting in any way. Embodiments of the elastic laminate 1500 may be used for side panels in pull-up diapers, such as training pants or adult incontinence products, or any other wearable article that may benefit from having different stretch zones with at least one different stretch property for providing an improved fit to the wearer.

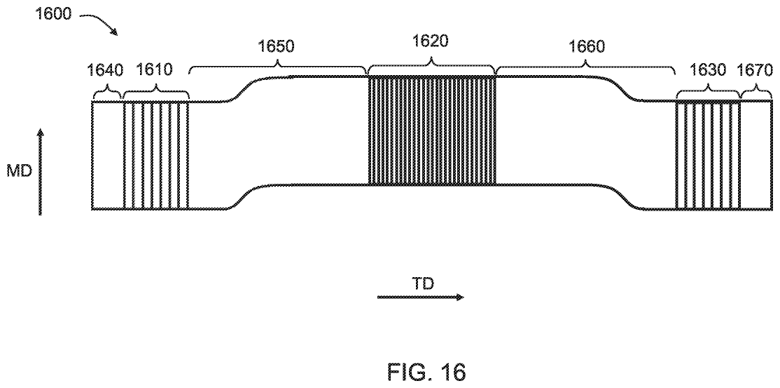

FIG. 16 is a schematic illustration of an elastic laminate 1600 in accordance with an embodiment of the invention. The elastic laminate 1600 includes a first stretch zone 1610, a second stretch zone 1620, and a third stretch zone 1630. In an embodiment, the first stretch zone 1610 and the third stretch zone 1630, may have the same stretch properties, such as the same level of extensibility, while the second stretch zone 1620, may have different stretch properties, such as a different level of extensibility than the first stretch zone 1610 and the third stretch zone 1630 when the elastic laminate 1600 is stretched in the transverse direction TD. In an embodiment, the second stretch zone 1620 may have a higher level of extensibility, for example, than the first stretch zone 1610 and the third stretch zone 1630. In an embodiment, the second stretch zone 1620 may have a lower level of extensibility, for example, than the first stretch zone 1610 and the third stretch zone 1630.

As illustrated in FIG. 16, a first inelastic zone 1640 may be positioned at one edge of the elastic laminate 1600, a second inelastic zone 1650 may be positioned between the first stretch zone 1650 and the second stretch zone 1620, a third inelastic zone 1660 may be positioned between the second stretch zone 1620 and the third stretch zone 1630, and a fourth inelastic zone 1670 may be positioned at the other edge of the elastic laminate 1600. More or less stretch zones and/or inelastic zones may be created in the elastic laminate 1600. The illustrated embodiment is not intended to be limiting in any way. Embodiments of the elastic laminate 1600 may be used for a waist member in diapers, for example, or any other wearable article that may benefit from having different stretch zones with at least one different stretch property for providing an improved fit to the wearer.

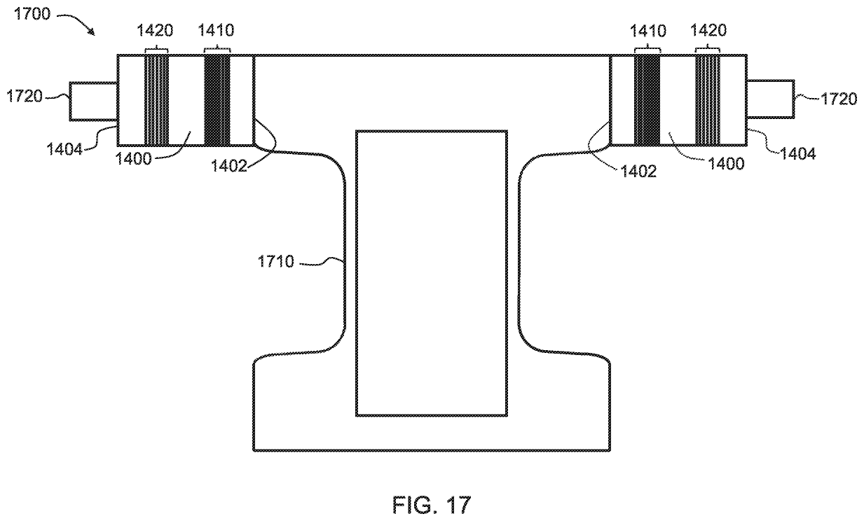

FIG. 17 is a schematic illustration of an absorbent article 1700 in the form of a diaper that includes a chassis 1710 and the elastic laminate 1400 of FIG. 14 used as ears attached to the chassis 1710 at the proximal end 402 thereof. A tab 1720, which may include one part of a hook and loop type fastener, is attached to the distal end 1404 of each elastic laminate 1400. The different stretch zones 1410, 1420 in the elastic laminate 1400 may be designed to provide the desired stretch properties along the elastic laminate 1400 to improve the fit for the wearer of the absorbent article 1700.

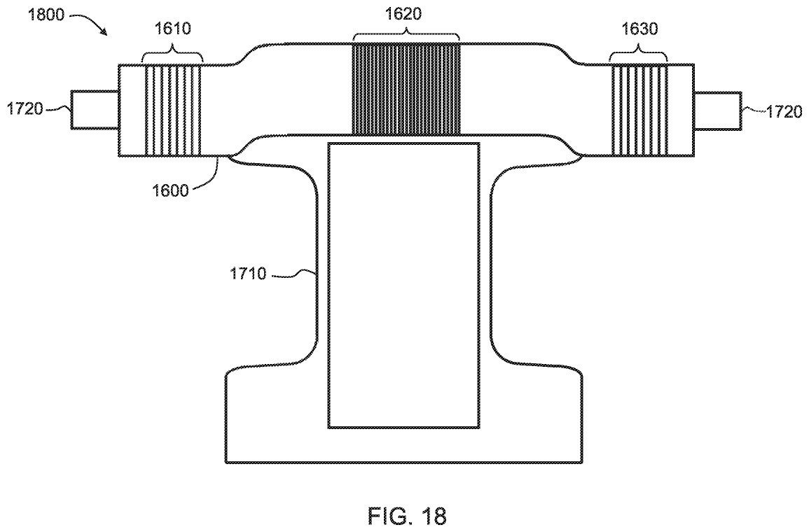

FIG. 18 is a schematic illustration of an absorbent article 1800 in the form of a diaper having the chassis 1710 and the tabs 1720 of FIG. 17, as well as the elastic laminate 1600 of FIG. 16. The different stretch zones 1610, 1620, 1630 and inelastic zones 1640, 1650, 1660, 1670 in the elastic laminate 1600 may be designed to provide the desired stretch properties along the elastic laminate 1600 to improve the fit for the wearer of the absorbent article 1800.

The embodiments described herein represent a number of possible implementations and examples and are not intended to necessarily limit the present disclosure to any specific embodiments. Instead, various modifications can be made to these embodiments, and different combinations of various embodiments described herein may be used as part of the invention, even if not expressly described, as would be understood by one of ordinary skill in the art. Any such modifications are intended to be included within the spirit and scope of the present disclosure and protected by the following claims.

What is claimed is:

1. A method for manufacturing an elastic laminate, the method comprising:
conveying an elastic laminate precursor material comprising an elastic film layer and a nonwoven layer in a machine direction to an activation station;
activating, at the activation station, a first zone of the elastic laminate precursor material to create a first stretch zone of the elastic laminate; and
activating a second zone of the elastic laminate precursor material to create a second stretch zone of the elastic laminate having at least one stretch property different from the first stretch zone of the elastic laminate.

2. The method according to claim 1, wherein the at least one stretch property is selected from the group consisting of: extensibility, modulus of elasticity, and permanent set.

3. The method according to claim 2, wherein the second stretch zone has a level of extensibility between about 10% and about 90% of a level of extensibility of the first stretch zone.

4. The method according to claim 3, wherein the level of extensibility of the second stretch zone is between about 20% and about 80% of the level of extensibility of the first stretch zone.

5. The method according to claim 4, wherein the level of extensibility of the second stretch zone is between about 30% and about 70% of the level of extensibility of the first stretch zone.

6. The method according to claim 1, wherein the first stretch zone and the second stretch zone extend in a direction transverse to the machine direction.

7. The method according to claim 1, wherein the second zone of the elastic precursor material is activated at the activation station.

8. The method according to claim 1, wherein the second stretch zone is adjacent the first stretch zone.

9. The method according to claim 1, wherein the first stretch zone and the second stretch zone are spaced apart by a third zone in a direction transverse to the machine direction, and wherein the third zone is not activated to create an inelastic zone in between the first stretch zone and the second stretch zone of the elastic laminate.

10. A method for manufacturing an elastic laminate, the method comprising:
conveying an elastic laminate precursor material comprising an elastic film layer and a nonwoven layer in a machine direction to a first activation station;
activating, at the first activation station, at least a portion of the elastic laminate precursor material to a first level of activation; and
activating, at a second activation station downstream in the machine direction from the first activation station, at least one zone of the elastic laminate precursor material to a second level of activation greater than the first level of activation to create at least two stretch zones of the elastic laminate having at least one stretch property different from each other.

11. An elastic laminate comprising:
an elastic film layer;
a nonwoven layer attached to a first surface of the elastic film layer;
a first stretch zone; and
a second stretch zone having at least one stretch property different from the first stretch zone.

12. The elastic laminate according to claim 11, wherein the at least one stretch property is selected from the group consisting of: extensibility, modulus of elasticity, and permanent set.

13. The elastic laminate according to claim 12, wherein first stretch zone has a first level of extensibility and the second stretch zone has a second level of extensibility, and wherein the second level of extensibility is between about 10% and about 90% of the first level of extensibility.

14. The elastic laminate according to claim 13, wherein the second level of extensibility is between about 20% and about 80% of the first level of extensibility.

15. The elastic laminate according to claim 14, wherein the second level of extensibility is between about 30% and about 70% of the first level of extensibility.

16. The elastic laminate according to claim 11, further comprising a second nonwoven layer attached to a second surface of the elastic film layer, opposite the first surface.

17. The elastic laminate according to claim 11, further comprising an inelastic zone in between the first stretch zone and the second stretch zone.

18. The elastic laminate according to claim 11, further comprising a third stretch zone having at least one stretch property different from the first stretch zone.

19. The elastic laminate according to claim 18, wherein the at least one stretch property is selected from the group consisting of: extensibility, modulus of elasticity, and permanent set.

20. The elastic laminate according to claim 19, wherein the first stretch zone has a first level of extensibility, the second stretch zone has a second level of extensibility, and the third stretch zone has a third level of extensibility, and wherein the third level of extensibility is different than the first level of extensibility and the second level of extensibility.

21. The elastic laminate according to claim 19, wherein the first stretch zone has a first level of extensibility, the second stretch zone has a second level of extensibility, and the third stretch zone has a third level of extensibility, and wherein the third level of extensibility is the same as the first level of extensibility or the second level of extensibility.

22. The elastic laminate according to claim 18, further comprising a first inelastic zone between the first stretch zone and the second stretch zone, and a second inelastic zone between the second stretch zone and the third stretch zone.

23. An absorbent article comprising:
a chassis; and
an elastic laminate attached to the chassis, the elastic laminate comprising an elastic film layer,
a nonwoven layer attached to a first surface of the elastic film layer, a first stretch zone, and
a second stretch zone having at least one stretch property different from the first stretch zone.

24. The absorbent article according to claim 23, wherein the elastic laminate is an ear.

25. The absorbent article according to claim 23, wherein the elastic laminate is a waist member.

26. The absorbent article according to claim 23, wherein the elastic laminate is a side panel.

27. The absorbent article according to claim 23, wherein the elastic laminate is continuous around a circumference of the absorbent article.
